(19) United States
(12) Patent Application Publication
(10) Pub. No.: US 2026/0158188 A1
(43) Pub. Date: Jun. 11, 2026

(54) BIOCOMPATIBLE POWDER-TYPE HEMOSTATIC AGENT AND METHOD FOR PRODUCING SAME

(71) Applicant: THERACION BIOMEDICAL CO., LTD., Seongnam-si (KR)

(72) Inventors: Hyun Kyoon KIM, Seongnam-si, Gyeonggi-do (KR); Eun Jin KIM, Seongnam-si, Gyeonggi-do (KR); Byung Nam KIM, Seongnam-si, Gyeonggi-do (KR); Hee Jung JO, Seongnam-si, Gyeonggi-do (KR)

(73) Assignee: THERACION BIOMEDICAL CO., LTD., Seongnam-si (KR)

(21) Appl. No.: 18/707,297

(22) PCT Filed: Oct. 17, 2022
(86) PCT No.: PCT/KR2022/015715

§ 371 Date: May 3, 2024

(30) Foreign Application Priority Data
Nov. 5, 2021 (KR) 10-2021-0151040

(51) Int. Cl. A61L 26/00; A61L 24/08

(52) U.S. Cl. CPC A61L 26/009; A61L 24/08; A61L 26/0023; A61L 26/0052; A61L 26/0066; A61L 2300/232; A61L 2300/236; A61L 2400/04

(57) ABSTRACT

Provided are a biocompatible powder-type hemostatic agent, which has high absorption and biological tissue adhesion, and a method for producing the same. More specifically, the biocompatible powder-type hemostatic agent is characterized by including first particles that have high blood absorption and second particles that have biological tissue adhesion, wherein the first particles and the second particles are bound to each other through a binder such that the hemostatic agent is in the form of a powdered granular formulation.

### TECHNICAL FIELD

The present invention relates to a biocompatible powder-type hemostatic agent having high absorption and biological tissue adhesion and a method for producing the same. More specifically, the biocompatible powder-type hemostatic agent includes first particles with high blood absorption and second particles with biological tissue adhesion, wherein the first particles and the second particles are bound to each other through a binder such that the biocompatible powder-type hemostatic agent has a powdered granular formulation.

### BACKGROUND ART

Hemostasis that is performed by controlling bleeding during surgery may reduce the possibility of a blood transfusion, shorten surgery time, and reduce postoperative complications, which improves patient safety and convenience. Therefore, various hemostasis methods are provided depending on the amount of bleeding during surgical operations.

In this regard, products that are commercialized as local hemostatic agents use oxidized regenerated cellulose, polysaccharides, collagen, gelatin, etc. and are provided in various forms, for example, a patch (sheet) form such as sponge or film, a fabric form such as fabric or non-woven fabric, or a colloid, paste, or gel form. In addition, there are products produced as a passive formulation that simply promotes blood absorption and an active formulation that promotes blood coagulation cascade by using fibrinogen and thrombin, which are components derived from animal and human blood. Recently, powder-type hemostatic agents that may stop bleeding with a large surface area at a bleeding wound site and are convenient for applications to narrow and thin areas have also been used in the market, and thus, the powder-type hemostatic agents have an advantage of being applicable to minimally invasive surgery (MIS).

Meanwhile, Arista is a representative powder-type hemostatic agent that is currently available on the market. Arista is a powder-type hemostatic agent that is cross-linked with starch and has an advantage of providing hemostatic effects by quickly absorbing blood when in contact with blood. However, when powder is applied by using a spraying device, the powder may remain in air, or the powder may stick to a cannula of the spraying device and clog an inlet of the spraying device or may float in the upper layer of blood even after in contact with blood. Accordingly, there is a disadvantage in that rebleeding may occur during removing the residual hemostatic agents after hemostasis is completed.

While conducting research to solve the above problems, the inventors of the present application developed a biocompatible powder-type hemostatic agent including first particles with high blood absorption and second particles with biological tissue adhesion, wherein the first particles and the second particles were bound to each other through a binder such that the biocompatible powder-type hemostatic agent had a powdered granular formulation. The inventors of the present application completed the present invention by discovered that the powder-type hemostatic agent had high blood absorption and biological tissue adhesion.

In this regard, Korean Patent Publication No. 10-2007-0095870 discloses an absorbable hemostatic agent.

### DESCRIPTION OF EMBODIMENTS

### Technical Problem

The present invention has been made in an effort to solve the problems of the related art, and an object of the present invention is to provide a biocompatible powder-type hemostatic agent with high absorption and biological tissue adhesion.

In addition, an object of the present invention is to provide a method for producing the biocompatible powder-type hemostatic agent.

### Solution to Problem

As a technical means for achieving the above technical objectives, an aspect of the present invention provides a biocompatible powder-type hemostatic agent.

The biocompatible powder-type hemostatic agent includes first particles with high blood absorption and second particles with biological tissue adhesion, wherein the first particles and the second particles are bound to each other through a binder such that the biocompatible powder-type hemostatic agent has a powdered granular formulation.

The first particles may include a material selected from the group consisting of carboxymethyl starch, carboxylmethyl cellulose, carboxylethyl cellulose, hydroxymethyl starch, hydroxymethyl cellulose, hydroxyethyl cellulose, dextrin, dextran sulfate, alginic acid, hyaluronic acid, chitin, chitosan, gellan gum, glucan, beta-glucan, chondroitin sulfate, glycogen, maltodextrin, fructan, galectin, mannan, salts thereof, and combinations thereof.

The second particles may include a material selected from the group consisting of carboxylmethyl cellulose, alginic acid (Alg), hyaluronic acid, chitin, chitosan, gellan gum, glucan, pullulan, sodium trimetaphosphate (STMP), gelatin, collagen, elastin, keratin, fibroin, casein, glutenin, phaseolin, albumin, salts thereof, and combinations thereof.

The binder may include a material selected from the group consisting of poly(vinyl pyrrolidone) (PVP), polyvinylidene fluoride, methyl cellulose (MC), hydroxypropyl methylcellulose (HPMP), carboxymethyl cellulose, povidone, xanthan gum, starch, alginic acid or a salt thereof, chitosan, polymethyl methacrylate (PMMA), polyvinyl alcohol (PVA), polyethylene-polypropylene copolymer, colloidal silica, clay dispersion, and combinations thereof.

A weight mixing ratio of the first particles to the second particles may be 1:0.1-100.

The binder may be included in an amount of 1-30 parts by weight based on 100 parts by weight of the first particles and the second particles.

Sizes of the first particles and the second particles may each independently be 500 μm or less.

A size of the biocompatible powder-type hemostatic agent having the powdered granular formulation may be 50 μm to 1,000 μm.

The biocompatible powder-type hemostatic agent may have a tapped density of greater than 1.5 g/mL, a blood absorption rate of 3.0 g/g or more, a blood clot formation amount of 0.3 g or more, and an adhesion of 0.5 N or more.

In addition, another aspect of the present invention provides a method for producing a biocompatible powder-type hemostatic agent with a powdered granular formulation.

The method includes: preparing first particles with high blood absorption and second particles with biological tissue adhesion; preparing a paste by adding a binder solution to the first particles and the second particles and mixing the binder solution with the first particles and the second particles; and freeze-drying the paste.

### ADVANTAGEOUS EFFECTS OF DISCLOSURE

The biocompatible powder-type hemostatic agent according to the present invention includes the first particles with high blood absorption, and thus may control primary bleeding by quickly absorbing a large amount of blood upon contact with blood. In addition, the biocompatible powder-type hemostatic agent includes the second particles with biological tissue adhesion. Accordingly, since the second particles reach the wound bleeding site and form a blood clot to thereby form a physical hemostatic barrier and reduce the possibility of rebleeding, it may be possible to achieve stable hemostasis until the surgery and procedure are completed.

Furthermore, since the biocompatible powder-type hemostatic agent is bio-degraded in vivo after hemostasis is completed, the risk of inflammation and side effects may be reduced.

Moreover, the biocompatible powder-type hemostatic agent may have remarkable water (blood) absorption rate, blood clot formation amount, adhesion, and hemostatic performance and may also have remarkable biodegradation.

### BRIEF DESCRIPTION OF DRAWINGS

FIG. 1 is a schematic diagram illustrating a mechanism of hemostasis action of a biocompatible powder-type hemostatic agent according to an embodiment of the present invention.

FIG. 2 is a scanning electron microscope (SEM) image showing a biocompatible powder-type hemostatic agent with a granular form produced according to an embodiment of the present invention.

FIG. 3 is a graph showing an in vitro blood absorption of biocompatible powder-type hemostatic agents produced according to a comparative example and examples of the present invention.

FIG. 4 is a graph showing an in vitro hemostasis time of biocompatible powder-type hemostatic agents produced according to comparative examples and examples of the present invention.

FIG. 5 is a graph showing an in vitro blood clot formation amount of biocompatible powder-type hemostatic agents produced according to comparative examples and examples of the present invention.

FIG. 6 is a graph showing an in vivo hemostasis time of biocompatible powder-type hemostatic agents produced according to a comparative example and an example of the present invention.

FIG. 7 is a graph showing an in vivo blood clot formation amount of biocompatible powder-type hemostatic agents produced according to a comparative example and an example of the present invention.

FIG. 8 is an image showing a biodegradation process of a biocompatible powder-type hemostatic agent produced according to an embodiment of the present invention over time.

### MODE FOR INVENTION

Hereinafter, the present invention will be described in more detail. However, the present invention may be embodied in various different forms and is not limited by embodiments described herein, and the present invention is only defined by the claims to be described below.

In addition, the terms as used herein are only used to describe specific embodiments and are not intended to limit the present invention. The singular forms as used herein are intended to include the plural forms as well unless the context clearly indicates otherwise. In the specification of the present invention, the phrase “including a certain element” means “further including other elements” rather than excluding other elements unless otherwise stated.

A first aspect of the present application provides a biocompatible powder-type hemostatic agent.

The biocompatible powder-type hemostatic agent includes first particles with high blood absorption and second particles with biological tissue adhesion, wherein the first particles and the second particles are bound to each other through a binder such that the biocompatible powder-type hemostatic agent has a powdered granular formulation.

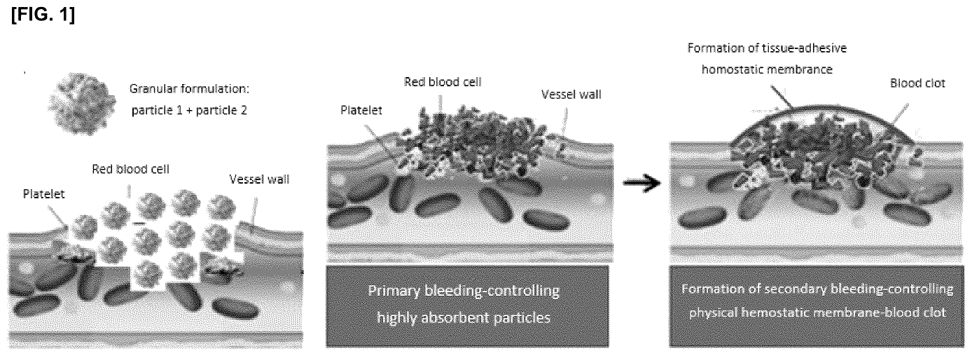

Hereinafter, the biocompatible powder-type hemostatic agent according to the first aspect of the present application will be described in detail with reference to FIG. 1.

In an embodiment of the present application, as illustrated in FIG. 1, the biocompatible powder-type hemostatic agent includes first particles with high blood absorption and second particles with biological tissue adhesion. Upon contact with blood, the biocompatible powder-type hemostatic agent may control primary bleeding by quickly absorbing a large amount of blood. In addition, the biocompatible powder-type hemostatic agent reduces the possibility of rebleeding because the second particles reach a wound bleeding site, generate a blood clot, and forms a physical hemostatic barrier. Therefore, it may be possible to achieve stable hemostasis until the surgery and procedure are completed. Furthermore, since the biocompatible powder-type hemostatic agent is degraded in vivo after hemostasis is completed, the risk of inflammation and side effects may also be reduced.

In an embodiment of the present application, the biocompatible powder-type hemostatic agent may include first particles with high blood absorption. In this case, the first particles may include a material selected from the group consisting of carboxymethyl starch, carboxylmethyl cellulose, carboxylethyl cellulose, hydroxymethyl starch, hydroxymethyl cellulose, hydroxyethyl cellulose, dextrin, dextran sulfate, alginic acid, hyaluronic acid, chitin, chitosan, gellan gum, glucan, beta-glucan, chondroitin sulfate, glycogen, maltodextrin, fructan, galectin, mannan, salts thereof, and combinations thereof. According to an embodiment of the present invention, carboxymethyl starch having a Ca²⁺-containing salt form, which is obtained by mixing carboxymethyl starch with CaCl₂), may be used.

In an embodiment of the present application, the first particles may have a water absorption rate of 20.0 g/g or more. According to an embodiment of the present invention, the first particles may have a water absorption rate of about 30.0 g/g. At this time, the water absorption rate may be calculated by Equation 1 below.

Water absorption (g/g)=(W2−W1)/W1 [Formula 1]
W1: initial weight (g) of sample
W2: weight (g) of sample having absorbed water

In an embodiment of the present application, the biocompatible powder-type hemostatic agent may include second particles with biological tissue adhesion. At this time, the second particles may include a material selected from the group consisting of carboxylmethyl cellulose, alginic acid (Alg), hyaluronic acid, chitin, chitosan, gellan gum, glucan, pullulan, sodium trimetaphosphate (STMP), gelatin, collagen, elastin, keratin, fibroin, casein, glutenin, phaseolin, albumin, salts thereof, and combinations thereof.

Meanwhile, according to an embodiment of the present invention, particles prepared by adding CaCl₂) to carboxymethyl cellulose and Na dissolved in water, particles prepared by adding CaCl₂) to alginic acid (Alg) and Na dissolved in water, particles prepared by adding CaCl₂) to a carboxymethyl cellulose/Na solution and an alginic acid (Alg)/Na solution dissolved in water, particles prepared by adding an alginic acid (Alg)/Na solution to a carboxymethyl cellulose/Na and CaCl₂) mixed solution, or
particles prepared by mixing pullulan with sodium trimetaphosphate (STMP) and then adding an alginic acid (Alg)/Na solution may be used as the second particles.

In an embodiment of the present application, the weight mixing ratio of carboxymethyl cellulose to alginic acid (Alg) may be 20:80 to 80:20. In this case, a content of Ca²⁺ may be 100 relative to the weight mixing ratio.

In an embodiment of the present application, the second particles may have an adhesion of 0.05 N or more, and the blood clot formation amount may be 0.150 g or more.

In an embodiment of the present application, the weight mixing ratio of the first particles to the second particles may be 1:0.1-100, and preferably 1:0.1-10. In addition, the sizes of the first particles and the second particles may each independently be 500 μm or less and may be sortable through a grinder and sieving.

In an embodiment of the present application, the binder may include a material selected from the group consisting of poly(vinyl pyrrolidone) (PVP), polyvinylidene fluoride, methyl cellulose (MC), hydroxypropyl methylcellulose (HPMC), carboxymethyl cellulose, povidone, xanthan gum, starch, alginic acid or a salt thereof, chitosan, polymethyl methacrylate (PMMA), polyvinyl alcohol (PVA), polyethylene-polypropylene copolymer, colloidal silica, clay dispersion, and combinations thereof. According to an embodiment of the present invention, poly(vinyl pyrrolidone) (PVP) may be used as the binder.

In an embodiment of the present application, the binder may be included in an amount of 1-30 parts by weight based on 100 parts by weight of the first particles and the second particles.

In an embodiment of the present application, the size of the biocompatible powder-type hemostatic agent with the powdered granular formulation may be 50 μm to 1,000 μm. Preferably, the average size of the biocompatible powder-type hemostatic agent may be 200 μm to 300 μm. At this time, the size of the biocompatible powder-type hemostatic agent may be sortable through sieving or the like.

In an embodiment of the present application, the biocompatible powder-type hemostatic agent may have a tapped density of greater than 1.5 g/mL, a blood absorption rate of 3.0 g/g or more, a blood clot formation amount of 0.3 g or more, and an adhesion of 0.5 N or more. In other words, since the biocompatible powder-type hemostatic agent is excellent in both the blood absorption rate and the blood clot formation amount, the hemostatic effect may be maximized. In addition, since the adhesion is also excellent, the biocompatible powder-type hemostatic agent may smoothly adhere to biological tissue.

In addition, the in vitro hemostasis time is less than 600 seconds, which means that hemostasis may be achieved very quickly. Furthermore, since biodegradation is more than 60%, the risk of inflammation and side effects may also be reduced.

A second aspect of the present application provides a method for producing a biocompatible powder-type hemostatic agent with a powdered granular formulation.

The method may include preparing first particles with high blood absorption and second particles with biological tissue adhesion, preparing a paste by adding a binder solution to the first particles and the second particles and mixing the binder solution with the first particles and the second particles, and freeze-drying the paste.

Although detailed descriptions of parts overlapping the first aspect of the present application are omitted, the descriptions of the first aspect of the present application may be equally applied even when the descriptions thereof are omitted in the second aspect.

Hereinafter, a method for producing a biocompatible powder-type hemostatic agent according to a second aspect of the present application will be described in detail step by step.

First, in an embodiment of the present application, the method for producing the biocompatible powder-type hemostatic agent may include preparing first particles with high blood absorption and second particles with biological tissue adhesion.

In an embodiment of the present application, the first particles may include a material selected from the group consisting of carboxymethyl starch, carboxylmethyl cellulose, carboxylethyl cellulose, hydroxymethyl starch, hydroxymethyl cellulose, hydroxyethyl cellulose, dextrin, dextran sulfate, alginic acid, hyaluronic acid, chitin, chitosan, gellan gum, glucan, beta-glucan, chondroitin sulfate, glycogen, maltodextrin, fructan, galectin, mannan, salts thereof, and combinations thereof. According to an embodiment of the present invention, carboxymethyl starch having a Ca²⁺-containing salt form, which is obtained by mixing carboxymethyl starch with CaCl₂), may be used.

In an embodiment of the present application, the second particles may include a material selected from the group consisting of carboxylmethyl cellulose, alginic acid (Alg), hyaluronic acid, chitin, chitosan, gellan gum, glucan, pullulan, sodium trimetaphosphate (STMP), gelatin, collagen, elastin, keratin, fibroin, casein, glutenin, phaseolin, albumin, salts thereof, and combinations thereof.

Meanwhile, according to an embodiment of the present invention, particles prepared by adding CaCl₂) to carboxymethyl cellulose and Na dissolved in water, particles prepared by adding CaCl₂) to alginic acid (Alg) and Na dissolved in water, particles prepared by adding CaCl₂) to a carboxymethyl cellulose/Na solution and an alginic acid (Alg)/Na solution dissolved in water, particles prepared by adding an alginic acid (Alg)/Na solution to a carboxymethyl cellulose/Na and CaCl₂) mixed solution, or particles prepared by mixing pullulan with sodium trimetaphosphate (STMP) and then adding an alginic acid (Alg)/Na solution may be used as the second particles.

In an embodiment of the present application, the weight mixing ratio of carboxymethyl cellulose to alginic acid (Alg) may be 20:80 to 80:20.

In this case, a content of Ca²⁺ may be 100 relative to the weight mixing ratio.

Next, in an embodiment of the present application, the method for producing the biocompatible powder-type hemostatic agent may include preparing a paste by adding a binder to the first particles and the second particles and then mixing the binder with the first particles and the second particles. At this time, the mixing may be performed by adding the first particles and the second particles to a high shear mixer or a fluidized bed granulator and spraying the binder into the high shear mixer or the fluidized bed granulator.

In an embodiment of the present application, the weight mixing ratio of the first particles to the second particles may be 1:0.1-100, and preferably 1:0.1-10. In addition, the sizes of the first particles and the second particles may each independently be 500 μm or less and may be sortable through a grinder and sieving.

In an embodiment of the present application, the binder may include a material selected from the group consisting of poly(vinyl pyrrolidone) (PVP), polyvinylidene fluoride, methyl cellulose (MC), hydroxypropyl methylcellulose (HPMC), carboxymethyl cellulose, povidone, xanthan gum, starch, alginic acid or a salt thereof, chitosan, polymethyl methacrylate (PMMA), polyvinyl alcohol (PVA), polyethylene-polypropylene copolymer, colloidal silica, clay dispersion, and combinations thereof. According to an embodiment of the present invention, poly(vinyl pyrrolidone) (PVP) may be used as the binder.

In an embodiment of the present application, the binder may be included in an amount of 1-30 parts by weight based on 100 parts by weight of the first particles and the second particles.

Next, in an embodiment of the present application, the method for producing the biocompatible powder-type hemostatic agent may include freeze-drying the paste.

In an embodiment of the present application, an aggregate paste prepared before the freeze-drying may be sorted for size by using a sieve mesh and then freeze-dried.

In an embodiment of the present application, the freeze-drying may be performed at a temperature of −100° C. to −30° C. for 10 hours or more. Preferably, the freeze-drying may be performed at a temperature of about −80° C. to −40° C. for 24 hours or more.

Thereafter, the biocompatible powder-type hemostatic agent with the freeze-dried powdered granular formulation may be sorted for size through sieving. At this time, the size of the biocompatible powder-type hemostatic agent may be 50 μm to 1,000 μm. Preferably, the average size of the biocompatible powder-type hemostatic agent may be 200 μm to 300 μm.

Hereinafter, examples of the present invention will be described so that those of ordinary skill in the art may easily carry out the present invention. However, the present disclosure may be implemented in various different forms and is not limited to the embodiments described herein.

### PRODUCTION EXAMPLE. PREPARATION OF FIRST PARTICLES AND SECOND PARTICLES

### 1. Preparation of First Particles

A starch self-assembly based on an ion complex was prepared.

More specifically, carboxymethyl starch (CM-starch) containing Ca²⁺ was prepared by mixing carboxymethyl starch with CaCl₂) and performing washing and drying thereon.

### 2. Preparation of Second Particles

\textcircled{1} Second Particles (CMC-Na+Alg-Na/CaCl₂)) were Prepared by Mixing CMC-Na with Alg-Na and then adding CaCl₂) thereto.

At this time, the weight mixing ratio of CMC-Na, Alg-Na, and CaCl₂) was 1:1:1.

\textcircled{2} Second particles (CMC-Na+CaCl₂)/Alg-Na) were prepared by mixing CaCl₂) with CMC-Na and then adding Alg-Na thereto.

At this time, the weight mixing ratio of CMC-Na, CaCl₂), and Alg-Na was 1:1:1.

\textcircled{3} The second particles were prepared in the same manner as \textcircled{1}, except that the weight mixing ratio of CMC-Na, Alg-Na, and CaCl₂) in the second particles of \textcircled{1} was changed to 25:75:100.

\textcircled{4} The second particles were prepared in the same manner as \textcircled{1}, except that the weight mixing ratio of CMC-Na, Alg-Na, and CaCl₂) in the second particles of \textcircled{1} was changed to 50:50:100.

\textcircled{5} The second particles were prepared in the same manner as \textcircled{1}, except that the weight mixing ratio of CMC-Na, Alg-Na, and CaCl₂) in the second particles of \textcircled{1} was changed to 75:25:100.

\textcircled{6} The second particles were prepared in the same manner as \textcircled{1}, except that the weight mixing ratio of CMC-Na, Alg-Na, and CaCl₂) in the second particles of \textcircled{1} was changed to 20:80:100.

\textcircled{7} The second particles were prepared in the same manner as \textcircled{2}, except that the weight mixing ratio of CMC-Na, CaCl₂), and Alg-Na in the second particles of \textcircled{2} was changed to 25:100:75.

Hereinafter, the types and content ratios of the second particles are summarized in Table 1 below.

TABLE 1

| | | Weight |
|---|---|---|
| No. | Type | content ratio |
| 1 | CMC-Na + Alg-Na/CaCl₂ | 100:100:100 |
| 2 | CMC-Na + CaCl₂/Alg-Na | 100:100:100 |
| 3 | CMC-Na + Alg-Na/CaCl₂ | 25:75:100 |
| 4 | CMC-Na + Alg-Na/CaCl₂ | 50:50:100 |
| 5 | CMC-Na + Alg-Na/CaCl₂ | 75:25:100 |
| 6 | CMC-Na + Alg-Na/CaCl₂ | 20:80:100 |
| 7 | CMC-Na + CaCl₂/Alg-Na | 25:100:75 |

### Example 1. Production of Biocompatible Powder-Type Hemostatic Agent with Powdered Granular Formulation

The first particles prepared in the above production example and the second particles prepared by \textcircled{3} (CMC-Na+Alg-Na/CaCl₂), weight content ratio: 25:75:100) were added to a high shear mixer at a weight mixing ratio of 50:50.

Thereafter, poly(vinyl pyrrolidone) (PVP) was sprayed into the high shear mixer and mixed with the first particles and the second particles. At this time, the content of the binder added was 5 wt % based on the first particles and the second particles.

Next, the prepared paste was sorted for size by using a mesh and then freeze-dried, and the freeze-dried granular formulation was sorted for size again by using a sieve. In this manner, a biocompatible powder-type hemostatic agent was produced.

### Example 2. Production of Biocompatible Powder-Type Hemostatic Agent with Powdered Granular Formulation

A biocompatible powder-type hemostatic agent was produced in the same manner as in Example 1, except that the second particles prepared in \textcircled{6} (CMC-Na+Alg-Na/CaCl₂), weight content ratio: 20:80:100) were used as the second particles in Example 1.

### Example 3. Production of Biocompatible Powder-Type Hemostatic Agent with Powdered Granular Formulation

A biocompatible powder-type hemostatic agent was produced in the same manner as in Example 1, except that the second particles prepared in \textcircled{7} (CMC-Na+CaCl₂)/Alg-Na, weight content ratio: 25:100:75) were used as the second particles in Example 1.

### Example 4. Production of Biocompatible Powder-Type Hemostatic Agent with Powdered Granular Formulation

A biocompatible powder-type hemostatic agent was produced in the same manner as in Example 3, but a powdered granular formulation lot was different and the physical property values derived in the following experimental examples were analyzed somewhat differently.

### Example 5. Production of Biocompatible Powder-Type Hemostatic Agent with Powdered Granular Formulation

A biocompatible powder-type hemostatic agent was produced in the same manner as in Example 3, except that first particles and second particles were added at a weight mixing ratio of 80:20.

### Example 6. Production of Biocompatible Powder-Type Hemostatic Agent with Powdered Granular Formulation

A biocompatible powder-type hemostatic agent was produced in the same manner as in Example 3, except that first particles and second particles were added at a weight mixing ratio of 20:80.

### Comparative Example 1. Preparation of Arista

Arista, a widely used powder-type hemostatic agent, was purchased and prepared.

### Comparative Example 2. Preparation of First Particles

The first particle (carboxymethyl starch, CM-starch) prepared in the production example described above were prepared alone
as a powder-type hemostatic agent.

### Experimental Example 1. Measurement of Water Absorption of First Particles

The water absorption of the first particles prepared in the preparation example and the Arista of Comparative Example 1 was measured and shown in Table 2 below.

At this time, the water absorption was measured by using Equation 1 below.

$$Water absorptio n ( g / g ) = ( W 2 - W 1 ) / W 1 \qquad [ Equation 1 ]$$

W1: initial weight (g) of sample
W2: weight (g) of water-absorbed sample

TABLE 2

| | Comparative | Production Example |
|---|---|---|
| | Example 1 | (first particles) |
| Water absorption | 16.0 | 30.0 |
| rate (g/g) | | |

As shown in Table 2 above, it was confirmed that the first particles prepared according to the production example of the present invention had a superior moisture absorption rate, compared to the Arista of Comparative Example 1.

### Experimental Example 2. Measurement of Physical Properties of Second Particles

The tapped density, amount of blood clot formation, and adhesion of the second particles prepared in \textcircled{1} to \textcircled{10} of the preparation example and Comparative Examples 1 and 2 were measured and are shown in Table 3 below.

TABLE 3
In vitro physical property comparison table

| | Tapped | Blood clot | |
|---|---|---|---|
| | density | formation | Adhesion |
| No. | (g/mL) | amount (g) | (N) |
| Comparative | 1.50 | 0.229 ± 0.03 | 0.01 |
| Example 1 | | | |
| Comparative | 1.03 | 0.395 ± 0.03 | — |
| Example 2 | | | |
| Production | 2.83 | 0.185 ± 0.02 | 0.17 |
| Example \textcircled{1} | | | |
| Production | 3.5 | 0.176 ± 0.01 | 0.38 |
| Example \textcircled{2} | | | |
| Production | 3.7 | 0.281 ± 0.02 | 0.23 ± 0.04 |
| Example \textcircled{3} | | | |
| Production | 9.3 | Not | 0.65 ± 0.05 |
| Example \textcircled{4} | | measurable | |
| Production | 16.0 | Not | 1.30 ± 0.20 |
| Example \textcircled{5} | | measurable | |
| Production | 2.7 | 0.240 ± 0.04 | 0.44 ± 0.05 |
| Example \textcircled{6} | | | |
| Production | 3.3 | 0.371 ± 0.07 | 0.26 ± 0.02 |
| Example \textcircled{7} | | | |

As shown in Table 3 above, in the case of the hemostatic agents according to Comparative Examples 1 and 2, the adhesion was very low or was not measured. In contrast, in the case of the second particles prepared in \textcircled{1} to \textcircled{7}, It was confirmed that the second particles had excellent adhesion. In addition, It was confirmed that the amount of blood clot formation was also at an appropriate level.

### Experimental Example 3. Observation of Morphology of Biocompatible Powder-Type Hemostatic Agent

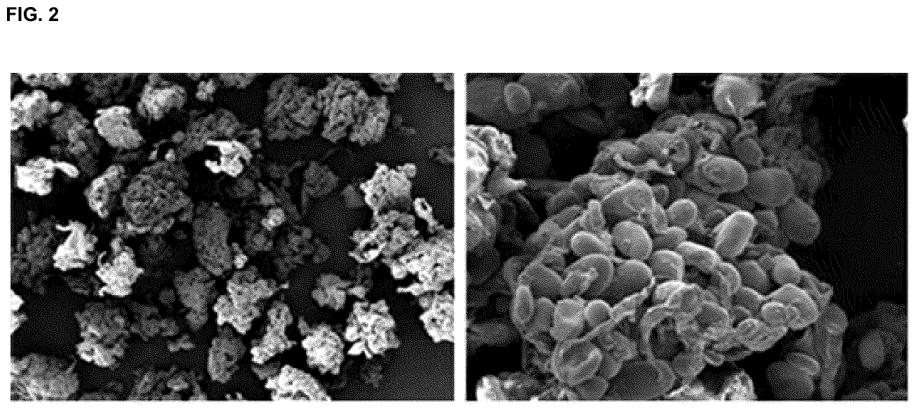

SEM images of the biocompatible powder-type hemostatic agent produced in Example 4 are shown in FIG. 2. As illustrated in FIG. 2, it was confirmed that the biocompatible powder-type hemostatic agent produced according to the embodiment of the present invention had a granular form in which the first particles and the second particles were agglomerated with each other by the binder solution, and voids were present therebetween.

### Experimental Example 4. Observation of Physical Properties of Biocompatible Powder-Type Hemostatic Agent

The physical properties of Arista prepared in Comparative Example 1 and the biocompatible powder-type hemostatic agents produced in Examples 1 to 3 were analyzed and are shown in Table 4 below.

TABLE 4
In vitro

| | Particle | Tapped | Blood clot | |
|---|---|---|---|---|
| | size | density | formation | Adhesion |
| No. | (μm) | (g/mL) | amount (g) | (N) |
| Comparative | 50-60 | 1.5 | 0.241 ± 0.03 | 0.64 ± 0.004 |
| Example 1 | | | | |
| Example 1 | 200-300 | 2.0 | 0.710 ± 0.03 | 0.97 ± 0.12 |
| Example 2 | 200-300 | 1.95 | 0.763 ± 0.05 | 1.12 ± 0.19 |
| Example 3 | 200-300 | 2.05 | 0.886 ± 0.03 | 1.02 ± 0.23 |

As shown in Table 4 above, it was confirmed that the biocompatible powder-type hemostatic agent produced according to the embodiment of the present invention had a larger particle size than the hemostatic agent of Comparative Example 1. In addition, it was confirmed that the amount of blood clot formation and adhesion were higher than the hemostatic agent of Comparative Example 1.

Furthermore, the physical properties of first particles prepared in Comparative Example 2 and the biocompatible powder-type hemostatic agents produced in Examples 4 to 6 were analyzed and are shown in Table 5 below.

TABLE 5
In vitro

| | Tapped | Blood clot | Blood | |
|---|---|---|---|---|
| | density | formation | absorption | Hemostasis |
| No. | (g/mL) | amount (g) | amount (g/g) | time (s) |
| Comparative | 1.03 ± 0.03 | 0.375 ± 0.002 | 3.90 ± 0.26 | 636 ± 56.7 |
| Example 2 | | | | |
| Example 4 | 2.22 ± 0.16 | 1.179 ± 0.02 | 10.9 ± 3.0 | 450 ± 18.4 |
| Example 5 | 2.20 ± 0.12 | 1.213 ± 0.06 | 11.3 ± 0.46 | 266 ± 42.9 |
| Example 6 | 2.33 ± 0.17 | 1.187 ± 0.01 | 8.8 ± 1.18 | 534 ± 72.4 |

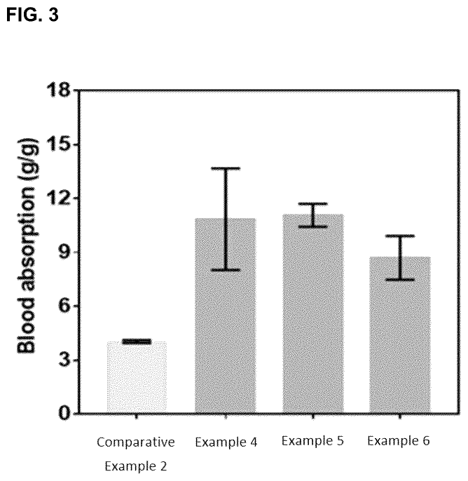

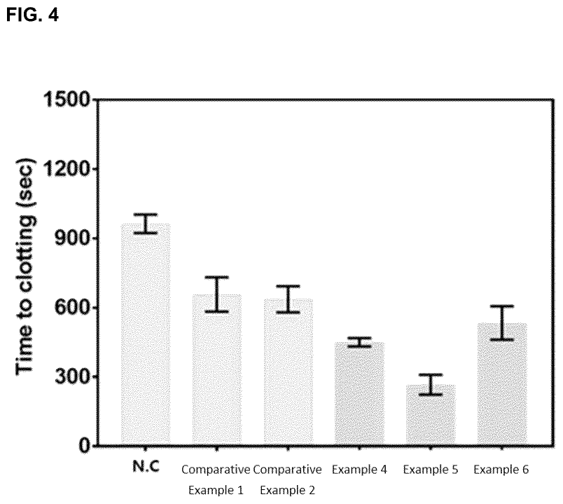

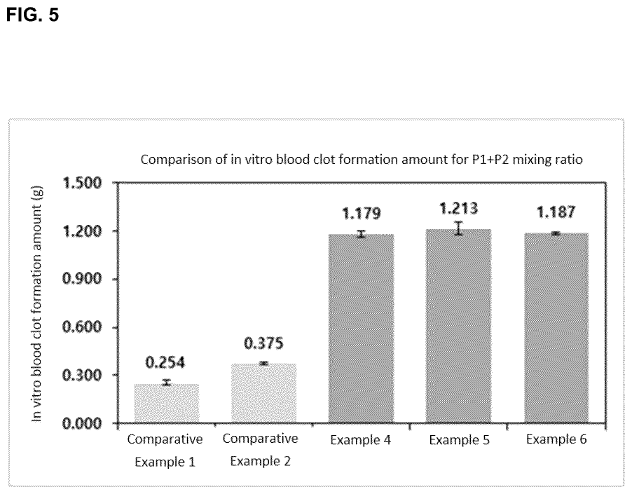

As shown in Table 5 above, it was confirmed that in the case of the biocompatible powder-type topographic agent produced according to the embodiment of the present invention, the tapped density, amount of blood clot formation, and blood absorption rate all increased compared to the hemostatic agent of Comparative Example 2 in which the first particles alone were included. Accordingly, it was confirmed that the hemostasis time was also significantly reduced (see FIGS. 3 to 5).

### Experimental Example 5. Observation of Biodegradation of Biocompatible Powder-Type Hemostatic Agent

The biodegradation of the biocompatible powder-type hemostatic agent produced in Examples 4 to 6 was measured by using Equation 2 below, and the results are shown in Table 6 below.

$$Degradation ( \% ) = \frac{{W}_{initial} - {W}_{Time point}}{{W}_{initial}} \times 100 ( \% ) \qquad [ Equation 2 ]$$

TABLE 6
In vitro

Biodegradation (%)

| Day | Example 4 | Example 5 | Example 6 |
|---|---|---|---|
| 0 | 0.00 | 0.00 | 0.00 |
| 1 | 83.5 ± 1.32 | 94.17 ± 0.58 | 59.83 ± 4.04 |
| 4 | 81.67 ± 0.58 | 94.17 ± 0.29 | 61.33 ± 4.07 |
| 7 | 84.5 ± 2.78 | 101.33 ± 0.29 | 60.67 ± 1.76 |

As shown in Table 6, it was confirmed that the biocompatible powder-type hemostatic agent produced according to the embodiment of the present invention exhibited excellent biodegradation.

### Experimental Example 6. In Vivo Hemostatic Performance Evaluation of Biocompatible Powder-Type Hemostatic Agent

In order to evaluate the in vivo hemostatic performance of Arista of Comparative Example 1 and the biocompatible powder-type hemostatic agents produced in Examples 4 to 6, a biopsy was performed on a rat liver at a depth of about 6 mm by using a 6-mm punch biopsy. Immediately after wound creation, blood was removed and the hemostatic agent was applied to each wound. Then, the hemostatic performance was evaluated.

The results thereof are shown in Table 7 below.

TABLE 7

| | Hemostasis | | Rebleeding |
|---|---|---|---|
| No. | time (s) | Deviation | (%) |
| Control | 228 | 83.3 | — |
| Comparative | 149 | 75.1 | 28.6 |
| Example 1 | | | |
| Example 4 | 39 | 28.3 | 0.0 |
| Example 5 | 49 | 33.1 | 0.0 |
| Example 6 | 32 | 18.9 | 0.0 |

As shown in Table 7 above, it was confirmed that in the case of the hemostatic agent produced according to the embodiment of the present invention, the hemostasis time was significantly reduced compared to Arista (Comparative Example 1). In addition, it was confirmed that hemostasis was achieved very well without rebleeding.

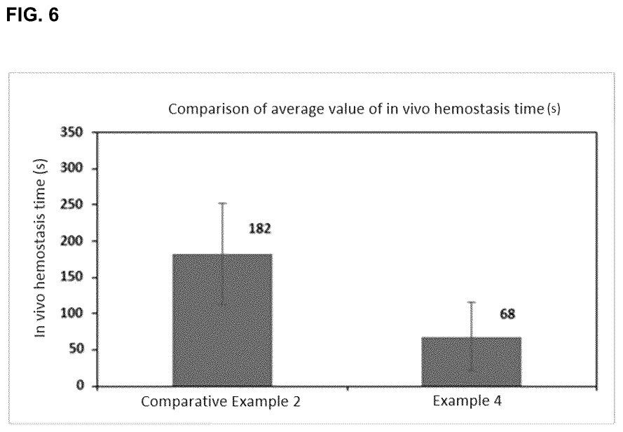

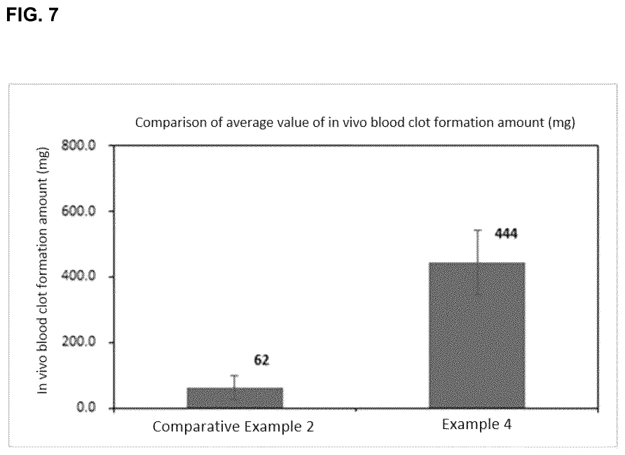

Furthermore, the punch biopsy was changed to 8 mm to induce more bleeding. The hemostatic performance of the hemostatic agent of Comparative Example 2 in which the first particles alone were included and the biocompatible powder-type hemostatic agent of Example 4 was evaluated in a method similar to the above, and the results thereof are shown in Table 8 below and FIGS. 6 and 7.

TABLE 8

| | | | Blood clot | |
|---|---|---|---|---|
| | Hemostasis | | formation | |
| No. | time (s) | Deviation | amount (mg) | Deviation |
| Comparative | 182 | 70.1 | 62 | 36.3 |
| Example 2 | | | | |
| Example 4 | 68 | 37.1 | 444 | 76.5 |

As shown in Table 8 above and FIGS. 6 and 7, it was confirmed that in the case of the hemostatic agent produced according to the embodiment of the present invention, the hemostasis time was significantly reduced and the amount of blood clot formation was also significantly increased, compared to the first particles of Comparative Example 2.

### Experimental Example 7. In Vivo Biodegradation Evaluation of Biocompatible Powder-Type Hemostatic Agent

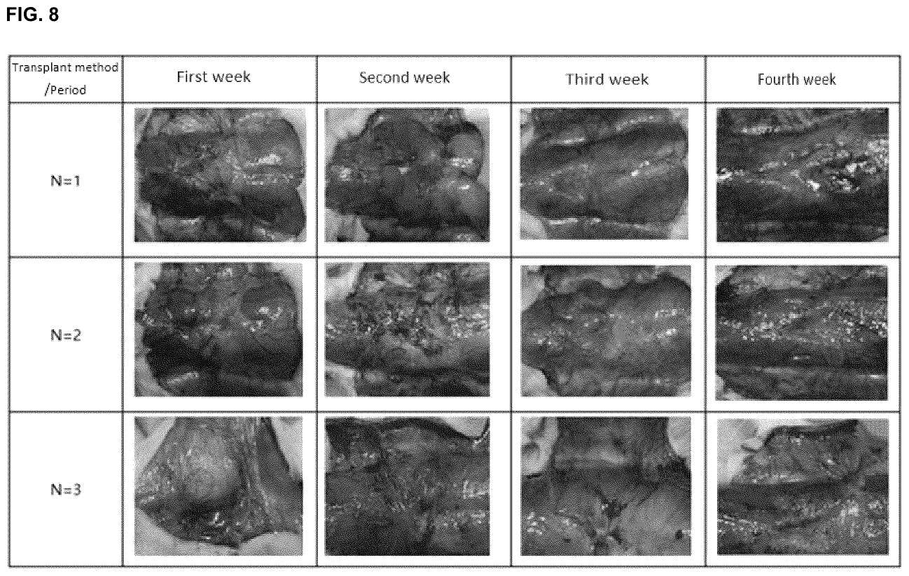

In order to evaluate the in vivo biodegradation of the biocompatible powder-type hemostatic agent produced in Example 4, after a rat's dorsal skin was incised, a hemostatic agent was inserted subcutaneously, and autopsies were performed at the first to fourth weeks. The results thereof are shown in FIG. 8.

As shown in FIG. 8, it was confirmed that the biocompatible powder-type hemostatic agent produced according to the embodiment of the present invention had excellent biodegradation because no hemostatic agent remained after the second week.

The present invention has been described in detail with reference to the preferred embodiments and the drawings, but the scope of the technical idea of the present invention is not limited to these drawings and embodiments. Accordingly, various modifications or equivalents thereof may fall within the scope of the technical idea of the present invention. Therefore, the scope of the technical idea according to the present invention should be interpreted by the claims, and the technical idea within the equivalents should be interpreted as falling within the scope of the present invention.

### INDUSTRIAL APPLICABILITY

The biocompatible powder-type hemostatic agent according to the present invention includes the first particles with high blood absorption, and thus may control primary bleeding by quickly absorbing a large amount of blood upon contact with blood. The second particles reach the wound bleeding site and form a blood clot to thereby form a physical hemostatic barrier and reduce the possibility of rebleeding. Therefore, it may be possible to achieve stable hemostasis until the surgery and procedure are completed. Furthermore, since the biocompatible powder-type hemostatic agent is degraded in vivo after hemostasis is completed, the risk of inflammation and side effects may be reduced. Moreover, the biocompatible powder-type hemostatic agent may be excellent in the water (blood) absorption rate, blood clot formation amount, adhesion, and hemostatic performance and may also have excellent biodegradation. Since the biocompatible powder-type hemostatic agent is useful when applied to the medical field, the biocompatible powder-type hemostatic agent has industrial applicability.

1. A biocompatible powder-type hemostatic agent comprising:
first particles with high blood absorption; and
second particles with biological tissue adhesion,
wherein the first particles and the second particles are bound to each other through a binder such that the biocompatible powder-type hemostatic agent is in the form of a powdered granular formulation.

2. The biocompatible powder-type hemostatic agent of claim 1, wherein the first particles comprise a material selected from the group consisting of carboxymethyl starch, carboxylmethyl cellulose, carboxylethyl cellulose, hydroxymethyl starch, hydroxymethyl cellulose, hydroxyethyl cellulose, dextrin, dextran sulfate, alginic acid, hyaluronic acid, chitin, chitosan, gellan gum, glucan, beta-glucan, chondroitin sulfate, glycogen, maltodextrin, fructan, galectin, mannan, salts thereof, and combinations thereof.

3. The biocompatible powder-type hemostatic agent of claim 1, wherein the second particles comprise a material selected from the group consisting of carboxylmethyl cellulose, alginic acid (Alg), hyaluronic acid, chitin, chitosan, gellan gum, glucan, pullulan, sodium trimetaphosphate (STMP), gelatin, collagen, elastin, keratin, fibroin, casein, glutenin, phaseolin, albumin, salts thereof, and combinations thereof.

4. The biocompatible powder-type hemostatic agent of claim 1, wherein the binder comprises a material selected from the group consisting of poly(vinyl pyrrolidone) (PVP), polyvinylidene fluoride, methyl cellulose (MC), hydroxypropyl methylcellulose (HPMP), carboxymethyl cellulose, povidone, xanthan gum, starch, alginic acid or a salt thereof, chitosan, polymethyl methacrylate (PMMA), polyvinyl alcohol (PVA), polyethylene-polypropylene copolymer, colloidal silica, clay dispersion, and combinations thereof.

5. The biocompatible powder-type hemostatic agent of claim 1, wherein a weight mixing ratio of the first particles to the second particles is 1:0.1-100.

6. The biocompatible powder-type hemostatic agent of claim 1, wherein the binder is included in an amount of 1-30 parts by weight based on 100 parts by weight of the first particles and the second particles.

7. The biocompatible powder-type hemostatic agent of claim 1, wherein sizes of the first particles and the second particles are each independently 500 μm or less.

8. The biocompatible powder-type hemostatic agent of claim 1, wherein a size of the biocompatible powder-type hemostatic agent with the powdered granular formulation is 50 μm to 1,000 μm.

9. The biocompatible powder-type hemostatic agent of claim 1, wherein the biocompatible powder-type hemostatic agent has a tapped density of greater than 1.5 g/mL, a blood absorption rate of 3.0 g/g or more, a blood clot formation amount of 0.3 g or more, and an adhesion of 0.5 N or more.

10. A method for producing a biocompatible powder-type hemostatic agent with a powdered granular formulation, the method comprising:
preparing first particles with high blood absorption and second particles with biological tissue adhesion;
preparing a paste by adding a binder solution to the first particles and the second particles and mixing the binder solution with the first particles and the second particles; and
freeze-drying the paste.
